(19) United States
(12) Patent Application Publication
(10) Pub. No.: US 2026/0230690 A1
(43) Pub. Date: Aug. 6, 2026

(54) APPARATUSES, SYSTEMS, AND METHODS FOR LED-BASED HYPERSPECTRAL IMAGING

(71) Applicant: OptoIntel, Inc., Cambridge, MA (US)

(72) Inventors: Lawrence Xinan CHEN, Cambridge, MA (US); Zachary KABELAC, Lexington, MA (US); Steven VOLLMER, Medford, MA (US); Mikael Marois, Cambridge, MA (US)

(73) Assignee: OptoIntel, Inc., Cambridge, MA (US)

(21) Appl. No.: 19/465,250

(22) Filed: Jan. 30, 2026

Related U.S. Application Data
(60) Provisional application No. 63/754,029, filed on Feb. 5, 2025.

(51) Int. Cl. H04N 23/12; H04N 23/55; H04N 23/56; H04N 23/60

(52) U.S. Cl. CPC H04N 23/12; H04N 23/55; H04N 23/56; H04N 23/665

(57) ABSTRACT

Apparatuses, systems, and methods for hyperspectral imaging are described. In certain implementations, a hyperspectral imaging system is provided. The system includes an illumination unit, an imaging unit, and a control unit in communication with the illumination unit and the imaging unit. The illumination unit includes a plurality of LEDs configured to emit light comprising at least one spectral band. The imaging unit includes an image sensor configured to capture an image of a target region of an imaging subject. The control unit performs synchronized control of the activation of the plurality of LEDs based on an activation pattern and the capture of images of the target region by the image sensor.

### CROSS-REFERENCE TO RELATED APPLICATIONS

This application is based on and claims benefit of priority of U.S. Provisional Patent Application No. 63/754,029, filed Feb. 5, 2025, the contents of which are incorporated herein by reference in its entirety.

### TECHNICAL FIELD

The present disclosure generally relates to the field of imaging, including apparatuses, systems, and methods for performing hyperspectral imaging. Some disclosed embodiments relate to, among other things, apparatuses, systems, and methods for hyperspectral retinal imaging with LED-based illumination.

### BACKGROUND

The eye is the window to the brain. It is the only part of the body with a direct view to blood vessels, veins, and arteries, especially on the brain side of the blood-brain barrier. This creates a unique opportunity to improve screening, diagnosis, and monitoring of brain disorders (e.g., dementia, Alzheimer's disease), neurological diseases (e.g., Parkinson's disease, stroke), cardiovascular diseases, retina disorders (e.g., diabetic retinopathy), and more. Systemic diseases can impact the eye through various pathways. Each of these pathways can be represented by unique spectral signatures. The eye is also a source of biomarkers for brain disorders, neurological diseases, cardiovascular diseases, retina disorders, etc. Current retinal imaging devices, such as retinal cameras, for capturing RGB retinal fundus images can be used to detect and diagnose various eye diseases. However, these devices are insufficient for distinguishing abnormal processes or key biomarkers from the background signal. Therefore, there is a need for apparatuses, systems, and methods for detecting and analyzing the unique spectral signatures of biomarkers, signals, or pathways for detecting, screening, diagnosing, and/or monitoring diseases.

### SUMMARY

Embodiments of the present disclosure are directed to devices, systems, and methods for performing hyperspectral imaging, including hyperspectral retinal imaging. Various embodiments of the disclosure may include one or more of the following aspects.

According to one exemplary aspect of the present disclosure, a hyperspectral imaging system is provided. The system includes an illumination unit, an imaging unit, and a control unit in communication with the illumination unit and the imaging unit. The illumination unit includes a plurality of light-emitting diodes (LEDs) configured to emit light comprising at least one spectral band. The illumination unit further includes a first optical assembly configured to receive the light from the plurality of LEDs and output a light beam comprising the at least one spectral band to illuminate a target region of an imaging subject. The imaging unit includes a second optical assembly configured to collect light reflected from the target region and direct the collected light to an image sensor. The imaging unit further includes an image sensor configured to capture an image of the target region. The control unit includes at least one processor and a non-transitory storage medium that stores instructions. The instructions, when executed by the at least one processor, cause the system to perform operations that include activating at least one LED of the plurality of LEDs based on an activation pattern. The activation pattern includes a selection of one or more LEDs. The one or more LEDs within a selection are to be activated simultaneously. The operations further include synchronizing the image capture of the target region and the illumination of the target region by coordinating exposure timing of the image sensor and the activation of the at least one LED.

Various embodiments of the hyperspectral imaging system may include one or more of the following features. In some embodiments, the activation pattern includes a time sequence of selections of one or more LEDs. The one or more LEDs within a selection are to be activated simultaneously. In some embodiments, the activation pattern includes a random or a pseudo-random selection of one or more LEDs from the plurality of LEDs. In some embodiments, the random or pseudo random selection of the plurality of the LEDs is based on a sparse sampling of a preselected spectral range. In some embodiments, the first optical assembly includes a spherical enclosure configured to distribute light emitted from the plurality of LEDs. In some embodiments, the spherical enclosure includes an inner reflective surface and an output aperture configured to output the light beam. In some embodiments, the spectral bands of one or more of the plurality of LEDs do not overlap, minimally overlap, or partially overlap.

In some embodiments, the operations include reconstructing a hyperspectral dataset of the imaging subject over a preselected spectral range. In some embodiments, the hyperspectral dataset includes a plurality of two-dimensional images corresponding to a plurality of wavelengths or spectral bands of the preselected spectral range. In some embodiments, the hyperspectral dataset includes a two-dimensional image for each wavelength or spectral bands of the preselected spectral range. In some embodiments, the operations further include measuring output power or intensity of the at least one LED activated based on the activation pattern. In some embodiments, the operations further include determining the reflectance spectral profile of the retina over the preselected spectral range based on the measured output power or intensity.

In some embodiments, the imaging subject includes at least a portion of the retina of an eye. In some embodiments, the operations include obtaining a hyperspectral profile of the portion of the retina over a preselected spectral range. In some embodiments, the operations further include detecting the presence or absence of at least one spectral signature in the hyperspectral profile indicative of the presence or absence of a biomarker in the retina. In some embodiments, the hyperspectral profile includes a reflectance spectral profile of the portion of the retina over the preselected spectral range.

According to another aspect of the present disclosure, a computer-implemented method for hyperspectral imaging is provided. The method includes activating at least one LED of a plurality of LEDs based on an activation pattern. The plurality of LEDs are configured to emit light comprising at least one spectral band. The activation pattern includes a selection of one or more LEDs to be activated simultaneously. The method includes capturing, by an image sensor, one or more images of a target region of an imaging subject under the illumination of the activated at least one LED. The method further includes synchronizing the image capture of the target region and the illumination of the target region by coordinating exposure timing of the image sensor and the activation of the at least one LED.

Various embodiments of the hyperspectral imaging system may include one or more of the following features. In some embodiments, the activation pattern includes a time sequence of selections of LEDs. The one or more LEDs within a selection are to be activated simultaneously. In some embodiments, the method includes performing a random or pseudo random selection of one or more LEDs from the plurality of LEDs for activation. In some embodiments, the method includes performing the random or pseudo random selection of the plurality of the LEDs based on a sparse sampling of a preselected spectral range. In some embodiments, the method includes distributing light emitted from the plurality of LEDs using a spherical enclosure. The spherical enclosure includes an inner reflective surface and an output aperture configured to output the light beam. In some embodiments, the spectral bands of one or more of the plurality of LEDs do not overlap, minimally overlap, or partially overlap.

In some embodiments, the method includes reconstructing a hyperspectral dataset of the imaging subject over a preselected spectral range. In some embodiments, the hyperspectral dataset includes a plurality of two-dimensional images corresponding to a plurality of wavelengths or spectral bands of the preselected spectral range. In some embodiments, the hyperspectral dataset includes a two-dimensional image for each wavelength or spectral band of the preselected spectral range. In some embodiments, the imaging subject comprises at least a portion of the retina of an eye. In some embodiments, the method includes obtaining a hyperspectral profile of the portion of the retina over a preselected spectral range. In some embodiments, the method includes detecting the presence or absence of at least one spectral signature in the hyperspectral profile indicative of the presence or absence of a biomarker in the retina. In some embodiments, the hyperspectral profile includes a reflectance spectral profile of the portion of the retina over the preselected spectral range. In some embodiments, the method includes measuring output power or intensity of LEDs activated based on the activation pattern. In some embodiments, the method includes determining the reflectance spectral profile of the portion of the retina over the preselected spectral range based on the measured output power or intensity.

Additional disclosure of the disclosed embodiments will be set forth in part in the description that follows.

It is to be understood that both the foregoing general description and the following detailed description are examples and explanatory only and are not restrictive of the disclosed embodiments as claimed.

The accompanying drawings constitute a part of this specification. The drawings illustrate several embodiments of the present disclosure and, together with the description, serve to explain the principles of certain disclosed embodiments as set forth in the accompanying claims.

### BRIEF DESCRIPTION OF DRAWINGS

FIG. 1 illustrates a hyperspectral imaging system for interrogating an imaging subject, consistent with embodiments of the present disclosure.

FIG. 2 is a schematic representation of an exemplary hyperspectral imaging system, consistent with embodiments of the present disclosure.

FIG. 3 is a schematic representation of exemplary activation patterns of a hyperspectral LED array, consistent with embodiments of the present disclosure.

FIG. 4 is a flowchart illustrating a method of synchronized control of illumination and image capture by an exemplary hyperspectral imaging system, consistent with embodiments of the present disclosure.

FIG. 5 is an example of using an exemplary hyperspectral imaging system to detect retinal biomarkers, consistent with embodiments of the present disclosure.

FIG. 6 is a flow diagram depicting a method for operating a hyperspectral imaging system, consistent with embodiments of the present disclosure.

### DETAILED DESCRIPTION

The present disclosure describes apparatuses, systems, and methods for hyperspectral imaging, such as for retinal imaging. In some embodiments, the apparatuses, systems, and methods use a programmable LED-based illumination unit, an imaging unit, and a control unit that synchronizes illumination and image capture. In some embodiments, a hyperspectral imaging system can operate in various modes, including single-band, multi-band, and sparse spectral sampling, to efficiently acquire high-resolution spatial and spectral data. Embodiments of the present disclosure are suitable for medical and diagnostic applications, such as detecting biomarkers and monitoring diseases like Alzheimer's, diabetic retinopathy, and age-related macular degeneration. Embodiments of the present disclosure are also applicable to non-diagnostic and non-medical uses. These applications include research on the optical properties of biological tissues or synthetic materials, material characterization, quality control, and other scientific or industrial applications where detailed spectral analysis is valuable.

Disclosed systems and methods address limitations of conventional imaging systems, such as insufficient spectral resolution, slow acquisition times, and inability to distinguish subtle molecular or structural features. By enabling rapid, high-fidelity hyperspectral illumination and imaging, the system provides solutions to address challenges related to molecular imaging, early disease detection, non-invasive monitoring, and comprehensive material and/or tissue analysis across clinical and non-clinical domains.

Reference will now be made in detail to embodiments and aspects of the present disclosure, certain examples of which are illustrated in the accompanying drawings. Where possible, the same reference numbers will be used throughout the drawings to refer to the same or like parts.

Consistent with some embodiments of the present disclosure, hyperspectral imaging is used to analyze the retina's molecular composition, which can provide insights into disease pathophysiology. In some embodiments, hyperspectral imaging is used to detect metabolic and/or vascular alterations by analyzing the spectral signatures of retinal layers. Taking Alzheimer's disease for example, Alzheimer's disease is associated with metabolic and vascular alterations in the brain, which can also manifest in the retina and detected by analyzing the spectral signatures of retinal layers.

In some embodiments, hyperspectral imaging can be used to detect amyloid beta (Aβ) plaques, a key biomarker of the disease, in the retina. Aβ plaques can accumulate in the retina of patients having Alzheimer's disease and could serve as a non-invasive biomarker for Alzheimer's disease diagnosis and monitoring. In some embodiments, hyperspectral imaging is used to detect spectral signatures associated with Aβ plaques, offering a potential method for early detection and monitoring of Alzheimer's disease progression. For example, beta-amyloid deposits or aggregates in the retina, particularly in the form of plaques, can result in different scattering properties compared to normal tissue, leading to a distinct spectral signature that can be detected and analyzed using embodiments of the present disclosure.

In some embodiments, hyperspectral imaging is used to analyze the relationship between Alzheimer's disease and ocular characteristics, providing new insights into disease mechanisms and potential therapeutic targets. As another example, Tau protein aggregation is another biomarker of potential Alzheimer's disease. Hyperspectral imaging can be used to detect tau-related changes in the retina, offering a non-invasive method for monitoring tau pathology.

Consistent with embodiments of the present disclosure, a hyperspectral retinal imaging system is provided for oculomics. The hyperspectral retinal imaging system provides spectral data that contains detailed molecular information, allowing for a deeper investigation of systemic diseases, especially those manifesting in the retina. Researchers can explore retinal vascular changes in diabetic retinopathy, identify metabolic shifts associated with age-related macular degeneration (AMD), and/or detect early signs of glaucoma by analyzing the retinal nerve fiber layer. By delivering highly granular, non-invasive imaging data, the hyperspectral retinal imaging system consistent with the present disclosure supports earlier disease detection and more personalized treatment strategies, ultimately improving patient outcomes.

While the present disclosure is described herein with reference to illustrative embodiments of certain applications, such as retinal imaging, fundus imaging, or Alzheimer's disease, it is understood that the embodiments described herein are not limited thereto. Those having ordinary skill in the art and access to the teachings provided herein will recognize additional modifications, applications, embodiments, and substitution of equivalents that all fall within the scope of the present disclosure. For example, disclosed systems and methods may be directed toward diagnostic methods or non-diagnostic and/or non-medical methods, such as imaging and detection methods of tissues or other materials for research or other non-medical methods. In some embodiments, hyperspectral imaging can be used for non-medical purposes, such as analyzing the optical properties of biological tissues or synthetic materials to study their composition, structure, and aging characteristics.

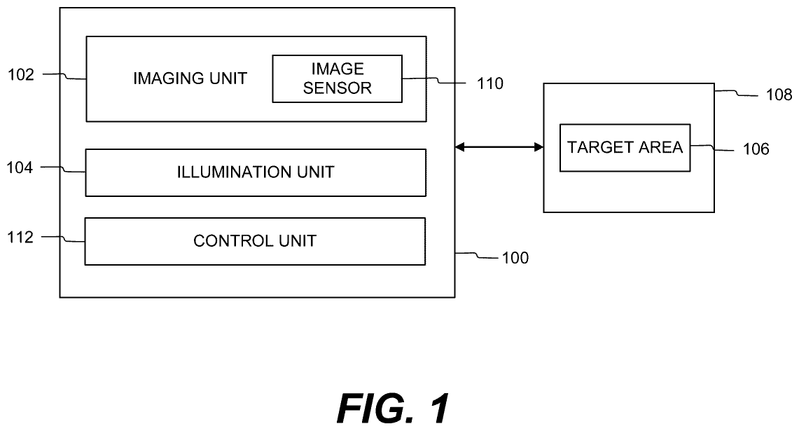

FIG. 1 illustrates a hyperspectral imaging system 100 including various components for imaging and/or detecting markers, such as biomarkers. In some embodiments, hyperspectral imaging system 100 includes an imaging unit 102 and an illumination unit 104. Hyperspectral imaging system 100 may include additional components, such as lenses, filters, and sensors. For example, imaging unit 102 and illumination unit 104 may use optical assemblies with various optical components, which may be used for independent or overlapping optical paths. Hyperspectral imaging system 100 is configured to interrogate an imaging subject 108 (e.g., retina, tissues, materials, etc.). In some embodiments, the interrogation includes detecting and/or analyzing a marker (e.g., a biomarker, a state of a biological pathway, a disorder, a disease, and/or a pathophysiological condition) in a target area 106 in imaging subject 108 (e.g., retina, tissues, materials, etc.).

A marker may refer to any characteristic, feature, or measurable element that indicates or signifies a particular state, condition, or process in a system, whether biological, chemical, physical, or otherwise. A biomarker may refer to a measurable indicator of a biological state or condition, often used to detect or monitor diseases, physiological processes, or responses to treatment. In some embodiments, hyperspectral imaging system 100 is configured to detect and/or analyze one or more spectral signatures using imaging unit 102 to detect the presence or absence of one or more markers in target area 106. Spectral signatures may refer to a spectral representation or pattern of absorption, reflection, transmission, or emission of electromagnetic radiation by a material (e.g., molecules or structures in the retina) over a range of wavelengths. A spectral signature may be associated with a unique feature of one or more markers in the spectral domain.

Imaging unit 102 may refer to an optical device that captures images or videos by focusing light onto a photosensitive image sensor, such as photodiode arrays, CCD (charge-coupled device) and CMOS (complementary metal-oxide-semiconductor) cameras. It should be understood that imaging unit 102 may include any type of image sensor for detection in hyperspectral imaging system 100. In some embodiments, imaging unit 102 is a hyperspectral camera. A hyperspectral camera may include an imaging device that captures images across one or more one or more spectral bands, spectral ranges, or ranges of wavelengths, producing spatial-spectral data that enables detailed analysis of the composition, structure, and properties of materials or scenes beyond what is possible with conventional color cameras.

In some embodiments, hyperspectral imaging system 100 is configured to obtain a hyperspectral image (or a hyperspectral dataset) using imaging unit 102, and a spectral profile is obtained for one or more pixels of the hyperspectral image. A spectral profile may refer to a representation of how the intensity or other properties of light vary across different wavelengths, providing characteristic information about the composition or state of a material, substance, or system. In some embodiments, a spectral profile spans across a plurality of wavelengths. In some embodiments, a spectral profile comprises a reflectance spectral profile. A reflectance spectral profile may refer to a measurement of how much light is reflected by a material or surface at each wavelength or spectral band across one or more spectral ranges, revealing information about its composition and properties. Other types of special profile may be obtained. In some embodiments, a spectral profile comprises an absorbance spectral profile. In some embodiments, a spectral profile comprises a fluorescence emission spectral profile. In some embodiments, imaging unit 102 is configured to capture spatial and spectral information indicating the presence or absence of a marker in target area 106.

In some embodiments, imaging unit 102 includes an image sensor 110 configured to capture spectral data. In some embodiments, image sensor 110 is configured to obtain the spectral information simultaneously in one capture or a single shot. For example, snapshot imaging can be performed by imaging unit 102. In snapshot imaging, one or more pixels on image sensor 110 of imaging unit 102 may be covered with a spectral filter. The spectral filter may have a high transmission or allow more light to pass through for one or more spectral bands while attenuating others. As described herein, a spectra band may refer to a range of wavelengths within the electromagnetic spectrum. A spectral band can be characterized by a center wavelength and a bandwidth (e.g., full width half maximum (FWHM)). The spectral filter may allow light of specific wavelengths or spectral bands to pass through to individual pixels on the image sensor, creating a spatial-spectral cube in a single shot. A spatial-spectral cube may refer to a three-dimensional hyperspectral dataset that captures both spatial information (image coordinates) and spectral information (wavelengths) for each point in a scene, enabling detailed analysis of material composition and structure. Such data captured by imaging unit 102 may be stored in the spectral data. For example, to obtain a spatial-spectral cube, a “mosaic sensor,” which has a mosaic of very small spectral filters, is located directly on the image sensor so that certain pixels (such as a group of adjacent pixels) are sensitive to specific wavelengths or spectral band. In some instances, the spatial resolution of the spatial-spectral cube may depend on the size of the pixels and the spectral filters, and the spectral resolution may depend on the bandwidth of the spectral filters.

In some embodiments, snapshot imaging can be performed to capture specific spectral bands in real time, making it suitable for real-time applications. Snapshot imaging may allow for obtaining a spatial-spectral cube in a single image capture. For example, snapshot imaging may be used for capturing dynamic scenes. Snapshot imaging may have a specific or limited spatial resolution since each pixel of the image sensor of imaging unit 102 may be configured to correspond to a specific wavelength or spectral band. In some embodiments, calibration of one or more spectral filters may be performed before image capture. Such calibration may use pre-established parameters or a reference phantom and/or other component with known optical properties. In some embodiments, alignment may be performed to align the spectral filters with the pixels of image sensor 110.

In some embodiments, illumination unit 104 includes a light source. In some embodiments, the light source may be a broadband light source that includes a UV light source, a visible light source, a near infrared light source, a visible and near infrared light source, such as a xenon or halogen lamp. It should be understood that any light source may be used with hyperspectral imaging system 100 depending on the use case, such as properties indicating a marker in target area 106.

Illumination unit 104 can be used for different imaging/illumination configurations, such as snapshot imaging and/or illumination sweep. In some embodiments, illumination unit 104 is configured to perform illumination sweeping such that the spectral information is obtained by sweeping the illumination wavelengths or illumination spectral bands. Illumination sweeping allows for decoupling spectral separation from image capture by imaging unit 102. In some embodiments, illumination unit 104 includes a tunable filter that sweeps across a plurality of wavelengths or spectral bands. In some embodiments, illumination sweeping is used to maximize camera pixel resolution. In some embodiments, a fast-sweeping filter is used for switching illumination wavelengths or spectral bands. When using a fast-sweeping filter, the camera's ability to capture spectral information may become limited by minimum exposure time to capture a quality image and the camera's frame rate and sensitivity. In some embodiments, illumination sweeping, such as by filtering or sweeping light wavelength or spectral band at the illumination side, not the camera side, may allow for minimizing the amount of light shown onto the eye. For example, the total amount of light of one wavelength or spectral band shown onto the eye among a sweeping range can be less than that of all wavelengths or spectral bands of the sweeping range shown onto the eye at the same time. This may reduce discomfort for the patient, for example.

In hyperspectral imaging system 100, various filtering techniques or various types of spectral filters may be used on the illumination and/or imaging sides, consistent with embodiments of the present disclosure. These spectral filters can include liquid crystal tunable filters (LCTF), acousto-optic tunable filters (AOTF), monochromators, and linear variable filters (LVF), each offering distinct advantages in terms of wavelength selection, speed, and spectral resolution. The choice of filter type allows the system to be tailored for specific applications, enabling precise control over the spectral bands captured and supporting both high-speed and high-fidelity hyperspectral imaging for detecting various markers in target area 106 or use cases. It should be understood that a spectral filter may include any combination of filters as discussed herein.

In some embodiments, a spectral filter may include a Liquid Crystal Tunable Filter (LCTF), which filters specific wavelengths by controlling the refractive index. LCTF can be inexpensive and widely available and covers a broad wavelength range. LCTF's switching speed from one wavelength or spectral band to another can be slow, such as within milliseconds or seconds, due to liquid crystal response times, but provide acceptable signal fidelity with low cost.

In some embodiments, a spectral filter may include an Acousto-Optic Tunable Filter (AOTF) that utilizes acoustic waves to diffract light selectively, offering tunability over a wide spectral range of the illumination light source. In some embodiments, AOTF's switching from one wavelength or spectral band to another can be fast, within microseconds, making it suitable for high-speed applications. AOTF also allows for high spectral resolution. AOTF can be expensive and may require complex optical configuration. Various factors, such as small filter area, narrow divergence angles, and polarization sensitivity, may need to be considered for alignment, calibration, and design choices.

In some embodiments, a spectral filter may include a Linear Variable Filter (LVF). LVF is a variable optical filter configured to be mechanically shifted in front of a prism or lens system to filter specific wavelengths or spectral bands. LVF can be flexible and allows for smooth transitions between spectral bands. LVF can provide similar functionality as a monochromator but with a more compact design. Using LVF may require precise movement and alignment, which may increase mechanical complexity of the system. Using LVF may not be preferred for high-speed applications due to the need to mechanically shift the filter, but in some instances, LVF may provide sufficient accuracy.

In some embodiments, a spectral filter may include a monochromator. In some embodiments, the monochromator includes a prism (or a grating) and a slit to select a specific wavelength or spectral band. The cost of using a monochromator can be moderate. Monochromators provide for precise spectral selection within a continuous spectral band. Monochromators can have limited bandwidth, as typically, only one continuous spectral band can be captured at a time. Thus, in some instances, using monochromators can be slower than snapshot imaging due to sequential spectral band capture, but they may provide advantages in accuracy and cost.

In some embodiments, hyperspectral imaging system 100 is configured with optimized spatial and spectral resolutions while minimizing the amount of light radiated into the patient's eye and/or reducing excess illumination. In some embodiments, hyperspectral imaging system 100 is configured to capture different spectral bands at various rates, allowing clinicians and researchers to tailor the operation to specific applications. For example, the system may be programmed to acquire one hundred spectral bands or wavelengths in a single second to detect biomarkers associated with Alzheimer's disease, such as amyloid beta, phosphorylated tau protein, or related retinal vascular and neural physiological alterations. For example, hyperspectral imaging system 100 can be configured to record three spectral bands at a rate of 30 Hz to monitor retinal oximetry as an indicator of blood flow. The versatility of hyperspectral imaging system 100 shortens the total acquisition time of traditional system while maintaining high spectral fidelity, reducing patient discomfort and increasing clinical throughput in retinal applications.

In some embodiments, hyperspectral imaging system 100 includes control unit 112. Control unit 112 may be a computer system including a processor and memory for operating hyperspectral imaging system 100. Control unit 112 may coordinate and synchronize the operation of illumination unit 104 and imaging unit 102, manage illumination patterns for the light source, control image acquisition timing, and process or store the acquired spectral data. In some embodiments, control unit 112 may also perform calibrations, execute reconstruction algorithms for generating hyperspectral images or datasets, and/or provide user interfaces for system configuration and data visualization.

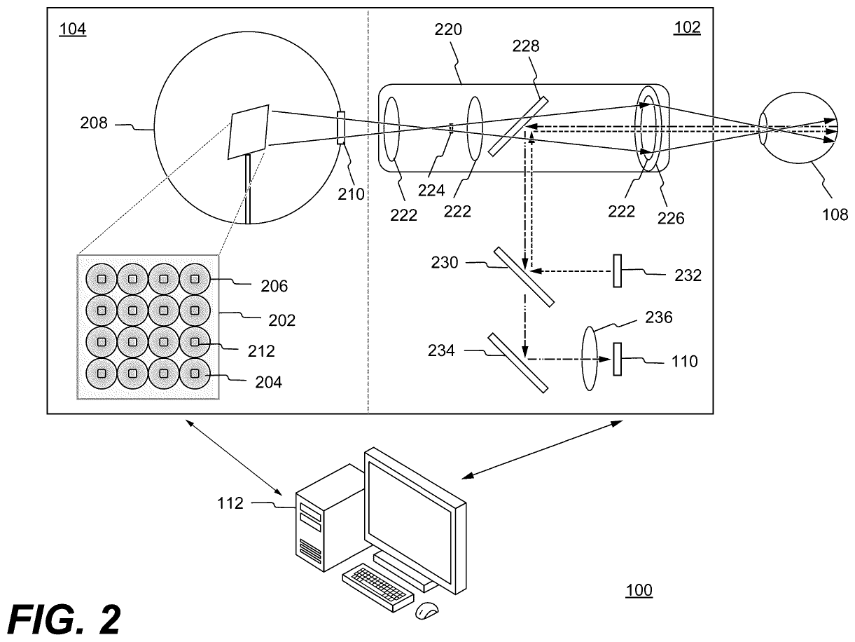

FIG. 2 is a schematic representation of hyperspectral imaging system 100 for imaging subject 108, consistent with embodiments of the present disclosure. In one example, imaging subject 108 is depicted as an eye with illumination targeting the retina on the back of the eye. As shown in FIG. 2, in some embodiments, hyperspectral imaging system 100 includes illumination unit 104 (e.g., a hyperspectral illumination subsystem) and imaging unit 102.

In some embodiments, illumination unit 104 includes a hyperspectral light-emitting diode (LED) array 202. Hyperspectral LED array 202 may be a compact, cost-effective illumination source (e.g., the light source described with respect to FIG. 1). Hyperspectral LED array 202 includes a plurality of LEDs 204. In some embodiments, hyperspectral LED array 202 covers the entire ultraviolet (UV) range or a portion of the UV range, such as from 320 nm to 400 nm. In some embodiments, hyperspectral LED array 202 covers the entire visible (VIS) range or a portion of the VIS range, such as from 400 nm to 750 nm. In some embodiments, hyperspectral LED array 202 covers the entire near-infrared (NIR) range of a portion of NIR range, such as from 750 nm to 1000 nm. In some embodiments, hyperspectral LED array 202 covers the entire remaining infrared (IR) range or a portion of the remaining IR range, such as from 1000 nm to 4300 nm.

In some embodiments, hyperspectral LED array 202 covers a combination of the UV, VIS, NIR, or IR ranges. For example, hyperspectral LED array 202 may include LEDs that have spectral bands spanning over a portion of the UV range and the entire or a portion of the VIS range, over the entire VIS range and the entire or a portion of the NIR range, over a portion of the UV range, the entire VIS range, and a portion of the NIR range, over a portion of the UV range, the entire VIS and NIR ranges, and a portion of the IR range, over the entire UV, VIS, and NIR ranges, or over the entire VIS, NIR, and IR ranges. As described herein, the LEDs of hyperspectral LED array 202 may be selected to have the spectral bands to continuously or intermittently span over a desired spectral range.

In some embodiments, each LED 204 in the grid is paired with a spectral filter 212 and a focusing lens 206. The spectral band of spectral filter 112 has a center wavelength and a spectral bandwidth corresponding to the pairing LED. For example, spectral filter 212 may have a center wavelength (the peak or midpoint of its spectral band) that matches or complements the emission spectral band of the corresponding LED. Spectral filter 212 may allow only light of the desired color or wavelength to pass through toward imaging subject 108. In some embodiments, spectral filter 212 may include a narrowband filter, such as a narrowband bandpass filter. The filter may also determine the spectral bandwidth, or the range of wavelengths around the center wavelength that are permitted to pass to the image sensor. A narrow bandwidth allows transmission of light very close to the center wavelength, resulting in high spectral resolution and more precise discrimination of spectral features. Conversely, a wider bandwidth permits a broader range of wavelengths, which can enhance signal strength but may reduce spectral specificity. The bandwidth and central wavelength of a spectral filter can be suitably selected for each use case.

In some embodiments, LEDs 204 of hyperspectral LED array 202 may include narrowband LEDs, with the number of LEDs 204 corresponding to the quantity of distinct spectral bands as needed for interrogating a target region. The number of LEDs 204 may depend on the desired spectral range for the hyperspectral dataset and the FWHMs of the spectral bands of the LEDs. For example, for a spectral range of 400-800 nm, hyperspectral LED array 202 may include eighty (80) LEDs each having a FWHM of about 5 nm or forty (40) LEDs each having a FWHM of about 10 nm. In some embodiments, each LED 204 may be paired with a narrowband spectral filter to further refine its spectral output and to reduce variability in spectral bandwidth among available LEDs 204. In some embodiments, quantum dot (QD) LEDs or bare LEDs may be used, and the narrowband spectral filters may be omitted. Hyperspectral LED array 202 may also include additional LEDs to support other device functions and imaging modalities, such as one or more broadband (white) LEDs for the imaging system, one or more infrared (IR) LEDs for alignment and focus tracking, and/or one or more LEDs for general illumination related for visualization of system components.

In some embodiments, each LED 204 may be individually controlled by and may be coupled to a power control circuit (e.g., a driver circuit controlled by control unit 112). In some embodiments, the power control circuit is designed to enable rapid switching at high power levels. Since the primary failure mode of LEDs may be thermal, operating each LED 204 for brief intervals (e.g., less than one second) may allow excitation at power levels significantly exceeding the rated continuous capacity, thereby achieving bright, narrowband illumination from compact LEDs. This approach may aid in the overall compactness and energy efficiency of illumination unit 104. The power control circuitry may be further engineered to provide fast response times for each LED 204, with steep ramp-up and ramp-down profiles to facilitate rapid hyperspectral scanning and to reduce unwanted spectral mixing between spectral bands. The power control circuitry may enable fully customizable operation, allowing a selected LED to be activated at desired levels of brightness, duration, and may enable simultaneous activation of multiple LEDs 204 within thermal constraints. By selectively activating multiple LEDs 204, custom spectral bands for illumination may be generated. In some embodiments, the spectral bands for simultaneous illumination have no overlap or minimum overlap (e.g., less than 5%). Dynamic control of illumination and imaging sensor exposure times also allows for differential exposure for specific bands as needed (e.g., higher exposure time for shorter wavelength ranges). This comprehensive and flexible control over the illumination environment allows for generation of custom hyperspectral scans optimized for screening markers (e.g., retinal biomarkers) associated with particular conditions or sets of conditions.

In some embodiments, hyperspectral imaging system 100 includes a calibration subsystem. The calibration subsystem may include an optical power meter and a spectrometer. A portion of the light output from illumination unit 104 is diverted via optical elements, such as mirrors, prisms, beamsplitters, and/or lenses, to the optical power meter and spectrometer for calibration. This calibration subsystem may continuously monitor the output of hyperspectral LED array 202 for both optical power and spectral purity, ensuring consistent and accurate illumination. In some embodiments, the calibration subsystem compares the measured output of hyperspectral LED array 202 to a predetermined value and may adjust the output accordingly for improved performance.

In some embodiments, hyperspectral LED array 202 may be arranged in a spherical enclosure 208, e.g., an integrating sphere, allowing for uniform light distribution through an output aperture 210 of the enclosure. As used herein, spherical enclosure 208 may refer to a type of enclosure with a highly reflective inner surface, designed to uniformly diffuse and distribute light for consistent illumination or measurement applications. Spherical enclosure 208 may allow light from each activated LED 204 to be evenly diffused and/or collected, which may minimize intensity hotspots and provide consistent illumination across the imaging subject, and enhance the quality and reliability of hyperspectral data acquisition.

In some embodiments, light from hyperspectral LED array 202 is directed from output aperture 210 to an optical assembly 220 in imaging unit 102. Optical assembly 220 may include lenses 222 for directing light from hyperspectral LED array 202 onto a desired target area of imaging subject 108. For example, optical assembly 220 may be configured with one, two, three, or more lenses 222 that receive, refract, or focus the light to the target area. In the example illustrated in FIG. 2, a total of three lenses 222 are used. Other suitable numbers of lenses may be used for different optical path designs. In some embodiments, other optical components may be used for defining the optical path, such as mirrors, prisms, and/or beam splitters.

In some embodiments, optical assembly 220 may include an annulus aperture 224. Annulus aperture 224 may contain a shaped mask and is configured to selectively block or shape traversing light. This prevents stray light reflecting from the target area of the imaging subject from reaching the image sensor. This may enhance image contrast and reduce stray light in the captured hyperspectral images, which may lead to improved image quality and resolution.

In some embodiments, optical assembly 220 may include an alignment ring 226 for aligning and calibrating illuminating light from hyperspectral LED array 202 directed to a target area of imaging subject 108. The alignment ring may incorporate NIR light sources that facilitate precise calibration and alignment, ensuring the illumination is accurately directed and uniformly distributed over the desired region for optimal hyperspectral image acquisition. In some embodiments, the NIR light source is used to perform eye tracking, which can help to perform broad alignment. In some embodiments, the NIR light source is used to illuminate the retina at a known wavelength, and a high framerate sensor can be used to capture images of the retina to perform fine alignment of the images in real time.

In some embodiments, optical assembly 220 may include additional components to allow flexible control over the optical path. In some embodiments, optical assembly 220 may include a beam director 228. Beam director 228 may be a circular mirror, prism, and/or beam splitter. The beam director can be configured to selectively direct, split, or modify the polarization state and/or direction of the illuminating or reflected light within optical assembly 220. For example, as shown in FIG. 2, beam director 228 receives and directs the light reflected from the target area towards an image sensor 110.

In some embodiments, when imaging subject 108 is the retina of a patient, optical assembly 220 includes one or more fixation targets 232. In some embodiments, imaging unit 102 may include a beam director 230 for directing some light between fixation targets 232 and imaging subject 108. In some embodiments, fixation targets 232 may include gaze fixation targets, such as one or more organic LED (OLED) fixation targets. Fixation targets 232 may provide precise, programmable visual cues to help align and stabilize the position of imaging subject 108 during image acquisition, improving image quality and repeatability. The integration of beam director 230 allows for dynamic control of the optical path and/or alignment, facilitating simultaneous imaging and subject fixation. Such configuration may be helpful for applications that would benefit from the imaging subject being steady during imaging, such as retinal imaging, where fixation targets 232 may assist the patient to maintain a steady gaze during imaging.

In some embodiments, imaging unit 102 may include mirror 234 for directing light through a focusing lens 236 onto image sensor 110. Image sensor 110 may be consistent with that described with respect to FIG. 1. The optical components in imaging unit 102 are positioned such that a desired target area of imaging subject 108 (e.g., where markers may be present) is imaged onto image sensor 110. In some embodiments, image sensor 110 is a high-speed camera sensor. In some embodiments, image sensor 110, alone or in combination with the optical components of hyperspectral imaging system 100, provides a high spatial resolution, such as equal to or less than 1 μm, 2 μm, 3 μm, 5 μm, or 10 μm. In some embodiments, image sensor 110, alone or in combination with the optical components of hyperspectral imaging system 100, provides a spectral resolution equal to or less than 5 nm, 8 nm, or 10 nm. The spatial and spectral resolution may be determined for one or more spectral bands over a 45-degree field-of-view, or over a larger field-of-view, such 100-degrees or 200-degrees. In some embodiments, with respect to retinal imaging applications, hyperspectral imaging system 100 may provide a total acquisition time that reduces or eliminates patient discomfort, such as equal to or less than 1 second, 1.5 second, 2 seconds, or 5 seconds. As described herein, the actual acquisition time may vary and depend on the particular use case, such as the imaging subject, the marker, the desired spectral range, or the illumination source.

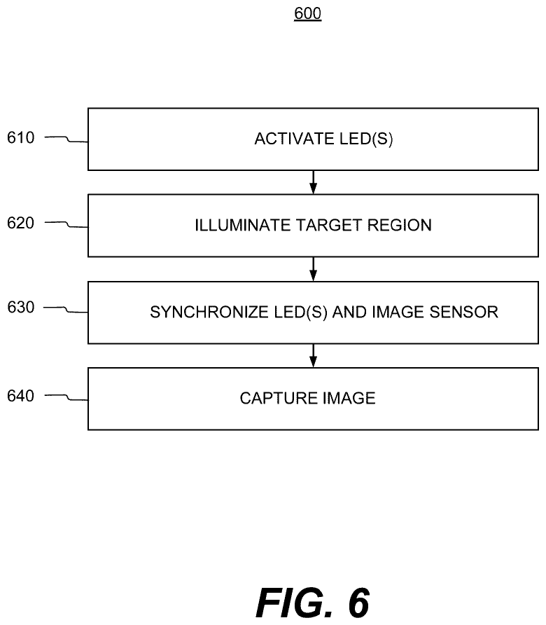

In some embodiments, hyperspectral imaging system 100 includes control unit 112 that is in communication with illumination unit 104 and imaging unit 102, such as by wired or wireless connection. In some embodiments, control unit 112 includes a computer system. In some embodiments, the computer system includes a user interface for controlling the system. The user interface may include various components for a user to interact with the system, such as a display (e.g., a touchscreen), a mouse, a keyboard, button, touchpad, thumbwheel, etc.), a status indicator (e.g., a display or light, a buzzer, or the like). In some embodiments, the computer system includes one or more processors, memory (e.g. RAM), buses that couple the processors and the memories, and non-volatile data storage device (e.g., ROM, hard disk drive, NVRAM), and a network interface. In some embodiments, a computer program is stored in the non-volatile data storage device and/or memory. The computer program may include computer executable code, when executed by the one or more processors, performs the steps of a method for operating hyperspectral imaging system 100. For example, a method for operating hyperspectral imaging system 100 is described below with reference to FIG. 6.

In some embodiments, hyperspectral LED array 202 is controlled and programmable by control unit 112. In some embodiments, control unit 112, such as a computer, communicates with illumination unit 104 and imaging unit 102 to enable synchronized capture of narrowband images across a plurality of wavelengths or spectral bands, such as UV, visible, and/or near-infrared (NIR) wavelengths or spectral bands. In some embodiments, hyperspectral LED array 202 is configured to emit over many narrow spectral bands. For example, hyperspectral LED array 202 may be configured to emit over a hundred spectral bands. Such computer controlled synchronized capture not only optimizes efficiency of data capture but also minimizes the exposure time for the patient. For example, by activating each LED 204 or group of LEDs 204 in coordination with control of image sensor 110 in imaging unit 102, hyperspectral LED array 202 can provide high-resolution, wavelength or spectral band specific illumination for hyperspectral imaging of imaging subject 108 and detection of markers.

In some embodiments, illumination unit 102 includes a control circuitry. In some embodiments, the control circuitry includes a power control circuit. In some embodiments, the control circuitry allows LEDs 204 to be driven at high power levels for short bursts and can switch on and off almost instantaneously, which allows hyperspectral imaging system 100 to excel in both speed and efficiency. For example, the control circuitry may turn a single LED 204 on and ramp up to full brightness over about 1 microsecond or less, maintain full brightness for an exposure time of several milliseconds, and then ramp down to 0% brightness in about 1 microsecond or less. In some embodiments, the control circuitry allows for synchronizing LED 204 activation with the camera shutter of image sensor 110, which helps reduce motion artifacts and improve capture of each spectral band image. In some embodiments, the control circuitry allows for controlling and programming hyperspectral LED array 202, such as allowing for optical multiplexing and/or uniform mixing of the outputs of a selection of LEDs 204.

In some embodiments, the compactness and/or efficiency of hyperspectral imaging system 100 can be improved by performing alignment and calibration before image acquisition. In some embodiments, baseline spectral images are obtained for calibration. In some embodiments, hyperspectral LED array 202 includes industry-standard LEDs. In some embodiments, hyperspectral LED array 202 includes medical-grade LEDs. In some embodiments, LEDs 204 of hyperspectral LED array 202 are a few millimeters in size, such as equal to or less than 5 mm, equal to or less than 4 mm, equal to or less than 2 mm. In some embodiments, hyperspectral LED array 202 can be expanded or modified to adapt to a desired spectral coverage, enabling a variety of applications and on-demand adaptations.

Consistent with the present disclosure, hyperspectral imaging system 100 may operate in one or more operational modes. In some embodiments, hyperspectral imaging system 100 operates in a single-band mode. In such single-band mode, only one LED (and thus one illumination spectral band) is activated at a time. Such an operational mode allows for straightforward single-band image acquisition. In some embodiments, hyperspectral LED array 202 may include N LEDs 204, each emitting a distinct spectral band. At any given time, one LED 204 is turned on, and image sensor 110 captures light at that spectral band. Thus, to capture spectral data spanning over N spectra bands, N successive illuminations and acquisitions are performed. This may simplify imaging and illumination conditions, allow for high fidelity control, and provide high-quality data.

In some embodiments, LEDs 204 of hyperspectral LED array 202 are activated based on the operational mode of hyperspectral imaging system 100. In some embodiments, the control circuitry of illumination unit 102 controls the activation of one or more LEDs 204 based on one or more activation patterns. In some embodiments, an activation pattern can be represented by an activation matrix A. Activation matrix A may refer to a structured representation that specifies, for each capture event, which LEDs 204 are turned on and which ones are off, typically using 1s and 0s to encode the illumination pattern.

For example, in single-band mode, activation matrix A can be a N×N matrix and used to describe LED 204 activation patterns over time, where N represents the number of spectral bands (e.g., LEDs 204). In this single-band mode, X is used to represent the reconstructed images per-band, and Y is used to represent the measured data (e.g., pixel data). In the single-band mode, activation matrix A is an Identity Matrix, with each row of A has one “1” (representing the active LED) and zeros “0” everywhere else (representing non-active LEDs). For example, activation matrix A is shown in the following equation that represents an exemplary use of the N×N matrix, where N=5 (e.g., A is a 5×5 identity matrix, representing 5 spectral bands and 5 images):

$$[ \begin{matrix}1 & 0 & 0 & 0 & 0 \\ 0 & 1 & 0 & 0 & 0 \\ 0 & 0 & 1 & 0 & 0 \\ 0 & 0 & 0 & 1 & 0 \\ 0 & 0 & 0 & 0 & 1\end{matrix} ] \times [ \begin{matrix}{X}_{1} \\ {X}_{2} \\ {X}_{3} \\ {X}_{4} \\ {X}_{5}\end{matrix} ] = [ \begin{matrix}{Y}_{1} \\ {Y}_{2} \\ {Y}_{3} \\ {Y}_{4} \\ {Y}_{5}\end{matrix} ] .$$

In single-band mode, the activation matrix A (e.g., the identity matrix) can be used to describe how spectral bands are combined in each captured image Y in response to activation of LED 204. The identity matrix has orthogonal rows (or columns), meaning each measurement is independent of the others and can be inverted without crosstalk. Because the activation pattern A corresponds to the identity matrix (A=I), the measured image data Y directly corresponds to the full set of images X. That is each row of X is just one captured image at a spectral band (AX=X=Y). In this mode, pixel intensities are explicitly associated with known emitted spectral bands in time because the system tracks which LED 204 was active during each image acquisition at a particular time. This makes the captured spectral data straightforward to organize, store, and analyze. For example, the first row in A indicates that a first LED is active, the second row in A indicates that a second LED is active, and so on. Xₙ represents the captured image when the n^{th} LED is active, and Yₙ represents the measured data corresponding to Xₙ captured when the n^{th} LED is active.

Single-band mode can be implemented by control unit 112 in a straightforward manner. Each image may correspond one-to-one with a spectral band. Single-band mode allows for high spectral resolution with minimal crosstalk because only one illuminating spectral band is active at a time, reducing or eliminating overlap between different spectral bands. Because the number of images captured is the same as the number of spectral bands, increasing the number of LEDs 204 or number of illumination spectral bands can increase the number of captured images for obtaining spectral data. In some cases, this could increase acquisition time and potential patient discomfort.

In some embodiments, hyperspectral imaging system 100 operates in more advanced operational modes. For example, in a multi-band mode, a group of selected LEDs 204 (e.g., a subset of the LEDs of LED array 202) are turned on to illuminate simultaneously to enable multi-band image acquisition. In some embodiments, an advanced operational mode, such as a sparse mode described below, use compressive sampling (or sparse sampling) techniques that leverage sparsity in received images (e.g., spectral sparsity in wavelength range of interest) to reduce the overall number and/or time of image acquisitions and improve patients' experience.

In some embodiments, hyperspectral imaging system 100 operates in a multi-band mode (e.g., a three-band RGB mode) to enable faster image acquisition. By activating at least one LED in each of the red, green, and blue bands simultaneously, multiple spectral bands are measured in a single capture, significantly reducing the number of captures compared to the single-band mode. This approach provides moderate spectral resolution with high spatial fidelity. For example, a three-band RGB mode, narrowband LEDs for red (R), green (G), and blue (B) are used to deliver different spectra for illumination, while an RGB camera of the imaging unit maintains its spatial resolution. Built-in color filters of the RGB camera naturally separate the R, G, and B channels, which simplifies data processing to extract spectral information. Activating LEDs in this configuration creates a diagonal activation matrix for each channel. This design balances acquisition speed and spectral fidelity, making it suitable for applications requiring moderate wavelength discrimination and high throughput.

In some embodiments, to reduce crosstalk, LEDs 204 with non-overlapping spectral bands (e.g., RGB) are selected and calibrated for each spectral band or color. Each LED 204 may include a dedicated spectral filter (e.g., a different spectral filter 212 for each LED 204 depending on the spectral band or color) to ensure narrowband illumination, and camera filters for red, green, and blue are calibrated according to the illumination spectral band or color for improved accuracy.

In multi-band mode using RGB LEDs (e.g., a three-band RGB mode), image sensor 110 has 3 different color channels that can be used to collect optical information (e.g., red, green, and blue channels). In some embodiments, three different LEDs 204 having separate spectral bands are used to illuminate a target region and image sensor 110 captures the light reflected from the target region, where each channel captures light from one of the three LEDs (e.g., by using a spectral filter). For example, each channel contains a built-in color filter (e.g., a Bayer pattern or similar color filter array), enabling the sensor to separate incoming light into red, green, and blue components. Additional spectral filters may be used to further ensure that, when a given LED is activated, the resulting signal is recorded primarily on the intended channel with minimal cross-channel contamination.

In multi-band mode, because each color channel effectively functions independently, the system may treat the red, green, and blue channels as three parallel acquisitions. The mapping between each LED 204 and its corresponding camera channel may be predetermined, based for example on the LED emission profile and the per-channel spectral response curves of image sensor 110. In this configuration, separate activation matrices may be established for the red, green, and blue channels, so that each channel can be described by its own illumination-to-measurement relationship. For example, the activation matrix A for the red channel may be defined over M image acquisitions as:

$$[ \begin{matrix}{A}_{{R}_{1}} & 0 & ⋯ & 0 \\ 0 & {A}_{{R}_{2}} & ⋯ & 0 \\ ⋮ & ⋮ & ⋮ & ⋮ \\ 0 & 0 & ⋯ & {A}_{{R}_{M}}\end{matrix} ] \times [ \begin{matrix}{X}_{{R}_{1}} \\ {X}_{{R}_{2}} \\ ⋮ \\ {X}_{{R}_{M}}\end{matrix} ] = [ \begin{matrix}{Y}_{{R}_{1}} \\ {Y}_{{R}_{2}} \\ ⋮ \\ {Y}_{{R}_{M}}\end{matrix} ]$$

where each value A_{R}_{₁}, A_{R}_{₂}, . . . A_{R}_{_{M}} takes a value 1 to indicate whether the red-band LED is active, and 0 if it is not. A similar formulation is applied for the green and blue channels, yielding corresponding activation matrices A_{G} and A_{B}. Accordingly, image sensor 110 may produce a set of three data streams, Y_{R}, Y_{G}, and Y_{B}, each representing the image data measurements acquired in its respective color channel. These measurement sets can then be expressed as:

$${Y}_{R} = {A}_{R} {X}_{R} , {Y}_{G} = {A}_{G} {X}_{G} , and {Y}_{B} = {A}_{B} {X}_{B} ,$$

where X_{R}, X_{G}, and X_{B} represent the reconstructed images associated with the red, green, and blue spectral bands, respectively. Because each channel contains measurements associated only with its assigned spectral region, the system can reconstruct band-specific image information for the RGB bands independently, enabling faster acquisition and reduced image count compared to single-band mode.

In some embodiments, hyperspectral imaging system 100 performs a sequential “sweep” across combinations of red, green, and blue LEDs 204 over a plurality of captures. In these embodiments, each image capture is carried out under illumination from a specific triplet of LEDs—one selected from the red band, one from the green band, and one from the blue band—such that the system systematically cycles through a defined set of RGB LED combinations. In some embodiments, the sweep may include all possible combinations of red, green, and blue LEDs, while in other embodiments a reduced or application-specific subset of combinations is used to optimize acquisition time or signal quality. Each combination of LEDs 204 may correspond to a row in activation matrix A, where the entries in that row specify which combination or LED triplet is active during that capture. Because each row of A defines one illumination condition, the complete matrix represents the full sequence of illumination states used during the acquisition. As the system iterates through these illumination conditions, it generates a corresponding series of image data measurements Y₁, Y₂, . . . , Y_{M}, where M denotes the total number of selected LED triplets. Each image data measurement Y_{M} may capture the reflected light resulting from the specific LED combination represented by the M-th row of A, thereby enabling channel-wise or joint reconstruction of image data using the known mapping between activation patterns of LEDs 204 and the capture image data.

Another advanced operational mode for operating hyperspectral imaging system 100 is a sparse mode. In some embodiments, the sparse mode leverages compressive sampling while activating only a subset of LEDs 204 in each capture, reducing redundancy and improving efficiency. In sparse mode, use of varying combinations of spectral bands can reduce the number of measurements required to detect and/or analyze markers, which may shorten the overall illumination time for the imaging subject.

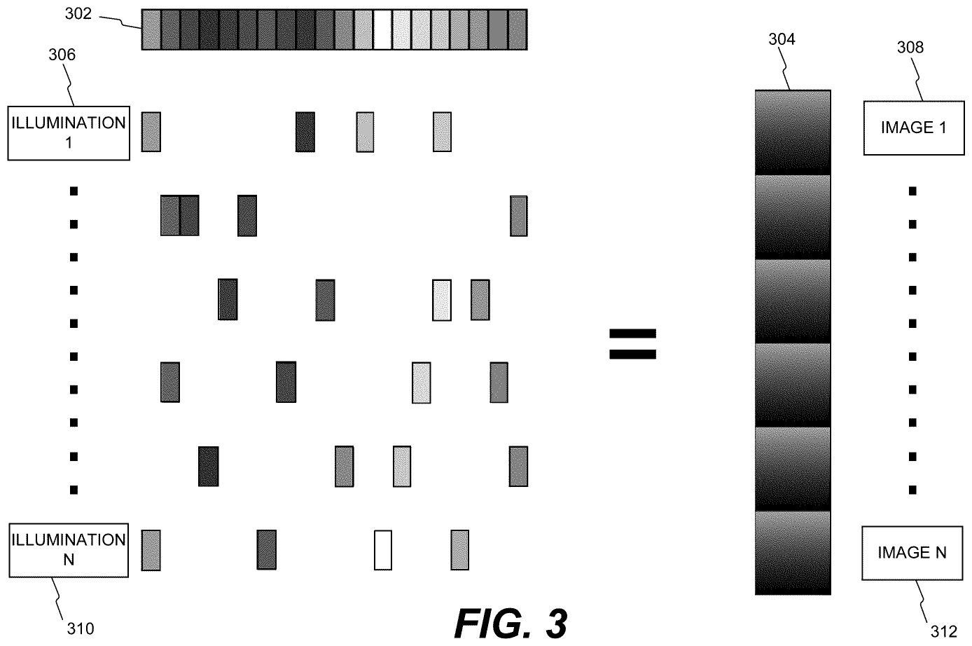

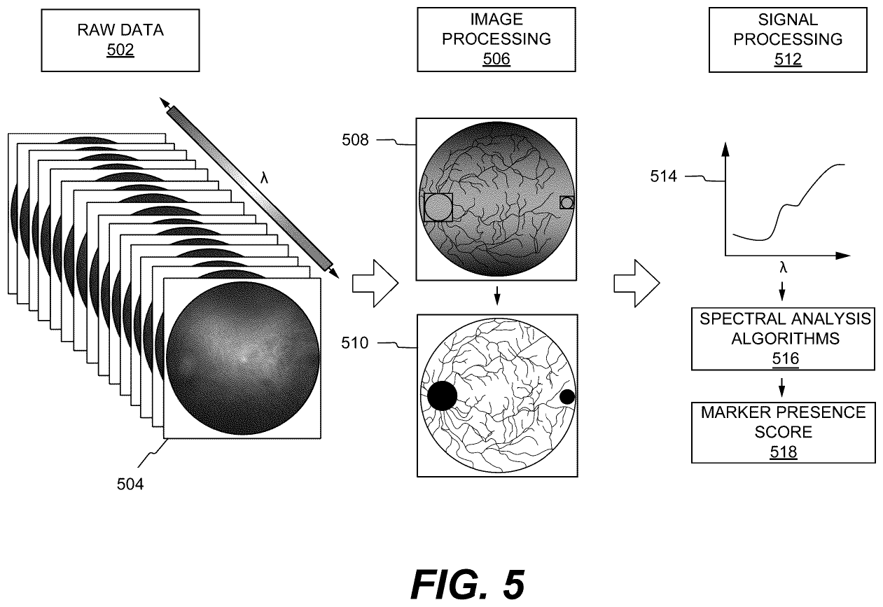

FIG. 3 is a schematic representation of exemplary activation patterns of a hyperspectral LED array (e.g., hyperspectral LED array 202 in FIG. 2), consistent with embodiments of the present disclosure. FIG. 3 incorporates by reference the components and operations of hyperspectral imaging system 100 as shown in FIGS. 1-2. FIG. 3 represents use of hyperspectral imaging system 100 in a sparse mode. In FIG. 3, spectral bands 302 are depicted in segments reflective of the spectral bands of individual LEDs (e.g., LED 204 in FIG. 2), which are used to illuminate the imaging subject and obtain an image set 304. In an illumination 1 (306), a set of four spectral bands 302 are illuminated. For example, a group of four LEDs may be activated at the same time. Upon illumination of the target area with the four LEDs, an image 1 (308) may be acquired, which may include imaged reflected light from a combination of the four spectral bands. Various activations of different LED combinations may be used for illuminating and acquiring each image in image set 304. Eventually, the illumination N (310) occurs, and image N (312) is obtained, indicating the end of the signal acquisition. After this, various post-processing and signal analysis may be performed, an example of which is further described with reference to FIG. 5.

In some embodiments, sparse mode leverages the flexibility of hyperspectral LED arrays to perform compressive spectral imaging, enabling the recovery of a full hyperspectral image or hyperspectral dataset from fewer measurements (e.g., fewer image captures) than the number of desired spectral bands over a preselected or desired spectral range. As used herein, compressive spectral imaging may refer to spectral reconstruction that applies compressive imaging, compressed imaging, compressive sensing, or compressed sensing techniques. For example, by simultaneously activating multiple LEDs 204 of different spectral bands (e.g., spectral bands 302), a combinatorial or mixing matrix of contributing spectra is produced and recorded in images (e.g., image 1 (308) . . . image N (312)). In some embodiments, LEDs 204 are activated based on a predetermined activation pattern that is generated as a pseudo-random sequence of one or more selected LEDs 204 to be simultaneously activated. This predetermined activation pattern yields a known mixing matrix of spectral contributions from the selected LEDs. Thus, under the sparse mode, instead of capturing one image per spectral band, the system acquires a set of combined or mixed images, e.g., “compressive measurements” or “compressed measurements” using multiple spectral bands per time of capture. The system may reconstruct the per spectral band image spanning a desired spectral range, such as spanning the VIS spectrum, the NIR spectrum, or both. In some embodiments, such sparse mode allows for determining the full hyperspectral cube with fewer measurements than the number of spectral bands (e.g., spectral bands 302) in a desired spectral range for the application.

In sparse mode, the matrix operations differ from single-band mode. If an image were captured at each spectral band individually, the per-band image matrix X would equal the image measurement matrix Y, as in single-band operation. In sparse mode, however, X represents the per-spectral band images to be reconstructed, while each entry in Y corresponds to a combined or mixed measurement resulting from the activation of multiple LEDs during a given capture, following different combinations of sparsely selected spectral bands. The activation matrix A encodes these sparse LED activation patterns, with each row containing one or more non-zero entries (e.g., the rows contain 0s and 1s) indicating which LEDs are active during each image capture. Each measured image data in Y represents a monochrome 2D image representing the summed contributions of the active LEDs 204 for that capture and recorded by an image sensor. The reconstruction of the hyperspectral dataset proceeds by solving for X in the following underdetermined linear system:

$$[ Y ] = [ A ] \times [ X ] .$$

In some embodiments, a hyperspectral dataset or hyperspectral images X over a spectral range is reconstructed. In some embodiments, a customized reconstruction algorithm is used. For example, an l1-minimization or basis pursuit technique may be used to recover X by exploiting spectral sparsity or compressibility of the reflectance spectrum at each pixel. In some embodiments, a reconstruction algorithm is designed and selected based on predetermined output intensities of LEDs 204 and the mixing matrix (e.g., activation matrix A). In some embodiments, calibration of the reconstruction algorithm is performed. In some embodiments, calibration is performed based on knowledge of LED outputs (e.g., measured output intensities or powers) and the combinatorial activation matrix A.

In some embodiments, additional reconstruction techniques may be used. For example, reconstruction techniques may include convex optimization methods, such as iterative shrinkage algorithms (e.g., FISTA) or alternating-direction methods (e.g., ADMM), greedy sparse-recovery algorithms, such as Orthogonal Matching Pursuit (OMP), Compressive Sampling Matching Pursuit (CoSaMP), or generalized OMP, and regularization methods. In some embodiments, low-rank, subspace-based, or tensor-decomposition methods may be used. In some embodiments, data-driven approaches, such as machine learning or deep-learning may be used.

Sparse mode is rooted in the principles of compressed sensing in signal processing where a high-dimensional signal is reconstructed from far fewer measurements than traditional sampling methods would require, provided the signal is sparse or compressible. In some embodiments, compressive measurements are performed to allow capturing sparse spectral samples, which can then be reconstructed into a full hyperspectral cube using compressed sensing algorithms. In some embodiments, the signal (e.g., hyperspectral information) is acquired in a basis different from the one in which it is sparse. Hyperspectral imaging system 100 may be configured to select these measurement bases, e.g., predetermined, and/or pseudo-random or random combinations of multiple spectral bands, the entire hyperspectral cube can be recovered from far fewer measurements than an exhaustive band-by-band sweep. Sparse mode allows for reducing the total image acquisitions required, minimizing imaging time and patient discomfort while preserving or even enhancing, diagnostic utility.

In some embodiments, activation of LEDs may follow a pseudo-random activation pattern to create a compressive sampling (or sparse sapling) process. In some embodiments, the pseudo-random activation pattern of the LEDs possesses robust mathematical properties. In some embodiments, images fewer than the number of illumination spectral bands are captured to reconstruct the hyperspectral dataset or hyperspectral image containing full spectral profiles of a target area of the imaging subject, such as the retina. In some embodiments, activation of LEDs follows a known mixing or combinational matrix. In some embodiments, various suitable computational techniques can be used to reconstruct per-spectral band images.

In some embodiments, sparse mode allows for design and implementing flexible and customizable LED activation patterns suitable for different applications. For example, LED activation patterns may be designed to optimize imaging for different clinical or research goals. In some embodiments, sparse mode allows for efficient data collection. For example, leveraging image sparsity, the total number of image acquisitions can be less than the total number of spectral bands, reducing total acquisition time and improving patient comfort.

In some embodiments, in sparse mode, hyperspectral imaging system 100 solves an underdetermined system of linear equations, where there are more unknowns (e.g., band-specific images X) than observed measurements (e.g., combined spectral images with summed contributions of multiple spectral bands Y), is solved. In order for a unique solution to exist, this problem is constrained by the principle of sparsity. In some embodiments, hyperspectral images obtained by the hyperspectral imaging system exhibit significant redundancy in both spectral and spatial domains. For example, while the retina may show variations in color and structure, these variations typically occur in a predictable or limited manner, allowing the data to be represented in a compressed basis. Such natural redundancy means there exists a sparse solution that fits the observed measurements, allowing for accurately recovering each spectral band's contribution despite the reduced number of captures. For example, selection of spectral bands 302 may be done retrospectively and/or may be predetermined. For retinal imaging, sparse mode operation may improve the determination of markers in the images while reducing the amount of light exposure to patients.

In the example of using hyperspectral imaging system 100 for retinal imaging, retinal images generally exhibit certain structural and spectral regularities. They show distinct, repeated features (e.g., blood vessels, optic disc, macula). Thus, in many transform domains (e.g., wavelets), these images can be represented efficiently. In addition, many biological tissues exhibit smooth, correlated reflectance spectra, which suggests that images at adjacent wavelengths can appear similar. While blood vessels are a prominent feature, the retina also contains multiple layers (e.g., nerve fiber layer, photoreceptor layer, etc.) and distinct regions (e.g., fovea, optic nerve head). Disease or retina states or conditions, such as diabetic retinopathy or AMD, can introduce additional details, lesions, or drusen, that change the apparent sparsity or complexity of the image. In some embodiments, the captured retinal images are not purely sparse. In some embodiments, the captured retinal images exhibit structural regularities and spectral correlations that can be exploited for compressed sensing.

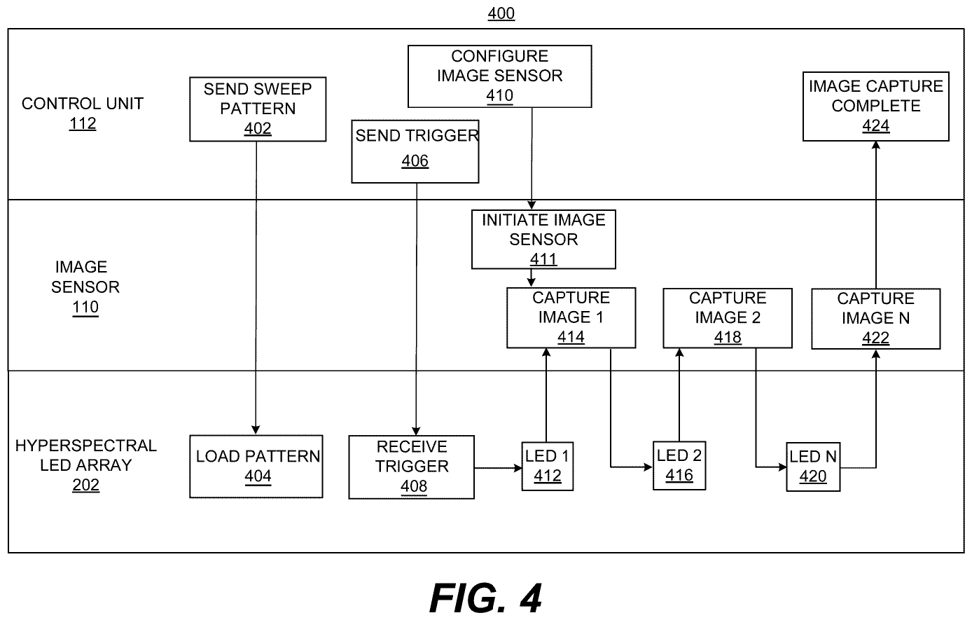

FIG. 4 is flowchart illustrating an example of a method 400 of synchronized control of illumination and image capture by an exemplary hyperspectral imaging system, consistent with embodiments of the present disclosure. FIG. 4 incorporates by reference the components of hyperspectral imaging system 100 in FIGS. 1-3, including control unit 112, image sensor 110, and hyperspectral LED array 202. Method 400 of FIG. 4 illustrates synchronized control of LEDs in hyperspectral LED array 202 and image sensor 110 in the context of a single-band mode, in which a single LED is switched on at each time of image capture by image sensor 110. The synchronized control can be performed in other operational modes of hyperspectral imaging system 100. For example, in a multi-band mode or a sparse mode, the activation of selected LEDs based on an LED activation pattern can be synchronized with image capture by image sensor 110.

In some embodiments, as shown in FIG. 4, an illumination sweep pattern is used for interrogating an imaging subject, such as for detecting one or more markers in the imaging subject (e.g., retina). In some embodiments, the illumination sweep pattern includes a time sequence of activation patterns, where each activation pattern represents a selection of one or more LEDs for activation at the same time. In step 402, control unit 112 may send an illumination sweep pattern to the illumination unit 104 including hyperspectral LED array 202. In some embodiments, the illumination sweep pattern is provided in the form of sweep parameters, such as parameters of an activation matrix and timing data. In step 404, a driver circuit of hyperspectral LED array 202 may then load the sweep parameters to initiate illumination sweep. In some embodiments, the sweep pattern may be configured by control unit 112 to follow a predetermined sequence of LED activation patterns depending on the target region and/or markers to be interrogated for the imaging subject. In some embodiments, step 404 may include preparing LEDs for activation and/or preloading operation parameters.

In some embodiments, step 406 includes triggering, by control unit 112, the start of the illumination sweep. In some embodiments, control unit 112 generates a trigger signal based on a user's instruction, which may be received via a user interface of control unit 112. In some embodiments, in step 410, control unit 112 retrieves or generates one or more configurations for the image sensor. In step 411, image sensor 110 receives the configurations from control unit 112 and prepares for image capture. Configurations for the image sensor 110 may include configuring the image sensor 110 to acquire images according to a particular timing (e.g., synchronized with activations of LEDs in hyperspectral LED array 202).

In some embodiments, in step 408, the driver circuit of hyperspectral LED array 202 receives the trigger signal from control unit 112, and in step 412 the driver circuit activates LED 1 to emit light of a first spectral band. In single-based mode, one LED of hyperspectral LED array 202 is activated. In multi-band mode or sparse mode, a selection of LEDs are activated.

In some embodiments, image sensor 110 is synchronized with LED activation. For example, in step 414, image sensor 110 may open its shutter upon activation of LED 1. The operation of the shutter of image sensor 110 may be controlled by control unit 112. For example, control unit 112 may send a control signal to image sensor 110 to open the shutter of image sensor 110 upon or shortly following the activation of an LED such that image capture occurs at about the same time upon the illumination of a target area by the LED of a spectral band. This synchronized illumination and image capture improves signal-to-noise for data acquired from a targeted area of an imaging subject.

In some embodiments, after image sensor 110 captures image 1 of a target region under the illumination of LED 1 in step 414, the shutter of image sensor 110 is closed and LED 1 is deactivated. Then, in step 416, LED 2 is activated and the shutter of image sensor 110 is opened to capture image 2 of the target region under the illumination of LED 2. Based on configurations established during steps 402, 404, and 410, hyperspectral LED array 202 and image sensor 110 may continue to operate in this fashion until in step 420, LED N, or a final LED or group of LEDs, has been activated, followed by step 422, where image sensor 110 captures image N of the target region.

In some embodiments, in step 424, after capture of image N has completed, control unit 112 may determine image capture complete, after which the system may initiate various post-processing events, including, but not limited to, image processing, data storage, spectral profile extraction, calibration verification, and preparation of the hyperspectral dataset for further analysis or visualization.

FIG. 5 is an example of using an exemplary hyperspectral imaging system 100 to detect retinal biomarkers, consistent with embodiments of the present disclosure. FIG. 5 incorporates by reference the components and operations of hyperspectral imaging system 100 as described with reference to FIGS. 1-4. As shown in FIG. 5, raw data 502 may be obtained. Raw data 502 may refer to raw images or pre-processed images, which may include images captured under single-band mode operation, multi-band mode, or sparse mode operation. For example, raw data 502 may be obtained using the operations shown in FIG. 4.

In some embodiments, raw data 502 may be obtained using single-band mode for LED illumination such that there is a 1:1 correlation between each spectral band and each captured image. In this mode, each image 504 in raw data 502 represents an image of a target area of an imaging subject, taken under the illumination of an activated LED with a particular central wavelength 2. For example, images 502 may be respectively captured under the illumination of LEDs having a central wavelength ranging from about 400 nm to about 765 nm, spanning VIS and NIR spectra. In this way, a stack of images 504 may be obtained with each image 504 corresponding to each activated LED. It should be understood that raw data 502 may include images corresponding to a plurality of spectral bands. Adjacent spectral bands may or may not have overlap.

In some embodiments, each raw image 504 may undergo image processing 506 for obtaining a hyperspectral dataset, which is then analyzed for detection of potential markers (e.g., biomarkers). Various types of image processing may be performed to each raw image 504 to obtain a corresponding pre-processed image 508, such as notice reduction, contrast enhancement, frame to frame registration, and spatial uniformity correction, intensity normalization or calibration. In some embodiments, image segmentation is performed on each pre-processed image 508 to identify features, such as retinal features (e.g., optic disc, fovea) and blood vessels in retinal images. In some embodiments, spectral information in the images can be leveraged for improved feature segmentation or contrast. For example, images captured at certain wavelengths may highlight hemoglobin in vessels and improve contrast with tissue in retinal images. In some embodiments, a mask, such as retinal mask, may be applied to each image 508 to enhance image contrast and improve visibility of relevant features, such as retinal features and vessels, and thereby generate a post-processed image 510. In some embodiments, the mask is automatically generated. Post-processed image 510 may have better signal-to-noise ratio for subsequent spectral analysis and detection of the markers of a target area.

In some embodiments, post-processed images 510 are used to obtain a hyperspectral dataset for further signal processing 512. For example, hyperspectral data 514 corresponding to at least a portion of the target region in post-processed images 510 may be obtained as a function of wavelength. In signal processing 512, this hyperspectral data (e.g., spectral data 108 in FIG. 1) may be further processed, such as noise filtering or spectral calibration. In some embodiments, the hyperspectral data is then analyzed, for example by using one or more spectral analysis algorithms 516 to generate a marker presence score 518. Marker presence score 518 may provide an indication of the presence of one or more spectral signatures indicative of the presence or absence of a marker in the target region. For example, marker presence score 518 may be a statistic value or probability derived from spectral analysis using spectral analysis algorithms 516. In some embodiments, spectral analysis algorithm 516 may include any suitable computational techniques for feature extraction and classification of hyperspectral data. Examples include spectral fitting methods, non-negative least squares (NNLS) methods, principal component analysis (PCA) methods, and other multivariate statistical or machine learning approaches. These algorithms may be configured to identify characteristic spectral patterns, reduce noise, and correlate spectral features with known markers, such as retinal biomarkers.

FIG. 6 is a flow diagram depicting a method 600 for operating a hyperspectral imaging system, consistent with embodiments of the present disclosure. The method may utilize any combination of the components of hyperspectral imaging system and/or operations as illustrated in FIGS. 1-5. The method pertains to synchronizing imaging and illumination units for obtaining hyperspectral images or datasets.

As shown in FIG. 6, in step 610 of method 600, one or more selected LEDs of an illumination unit are activated. Upon activation, each LED emits light with a specific center wavelength or spectral band, as defined by its characteristics and/or associated spectral filter. The sequential or simultaneous activation of different LEDs allows the system to build a comprehensive spatial-spectral dataset for subsequent hyperspectral analysis. In some embodiments, a control unit is used to establish activation patterns and/or control timing of LED activation. In some embodiments, activation patterns are established based on the operational mode of the system, such as a single-band mode, a multi-band mode, or a sparse mode.

In step 620 of method 600, light from the activated LED(s) is directed to a target area of an imaging subject for illumination and imaging. An optical assembly directs the emitted light onto the target area of the imaging subject, such as the retina or other ocular structures. This targeted illumination reduces the amount of light needed for imaging. The target area may then absorb, scatter, and/or reflect the light.

In step 630 of method 600, LEDs and shuttle of the image sensor are synchronized to allow the selected LED(s) to be activated in coordination with the image sensor's exposure timing, so that the image sensor captures images only when the desired spectral band is being emitted. By aligning the illumination and image acquisition cycles, the system reduces spectral crosstalk and increases the accuracy of the captured hyperspectral data.

In step 640 of method 600, images are captured using an image sensor in an imaging unit in the hyperspectral imaging system. In some embodiments, the image sensor receives the light reflected from the target area and captures an image of the target area. In some embodiments, the synchronized LED activation and image capture are performed repeatedly, with each image corresponding to a specific spectral band(s) as determined by the activation of the selected LEDs and/or the associated spectral filters. This process enables the acquisition of images with high spatial resolution and high signal-to-noise spectral data, where each pixel contains both spatial and spectral information. The captured images are then stored for subsequent signal processing and analysis, facilitating detailed examination of material composition and structure to identify markers, including biomarkers of potential diseases.

A computer-readable medium, for example, a non-transitory computer-readable medium, is provided. In some embodiments, the non-transitory computer-readable medium stores instructions for one or more processors of a control unit (e.g., control unit 112 as depicted in FIG. 1) for performing methods according to embodiments of the present disclosure. For example, the instructions stored in the non-transitory computer-readable medium may be executed by the circuitry of the control unit, the illumination unit, and/or the imaging unit for performing any of the above disclosed processes in part or in entirety. Common forms of non-transitory media include, for example, a floppy disk, a flexible disk, hard disk, solid-state drive, magnetic tape, or any other magnetic data storage medium, a Compact Disc Read-Only Memory (CD-ROM), any other optical data storage medium, any physical medium with patterns of holes, a Random Access Memory (RAM), a Programmable Read-Only Memory (PROM), and Erasable Programmable Read-Only Memory (EPROM), a FLASH-EPROM or any other flash memory, Non-Volatile Random Access Memory (NVRAM), a cache, a register, any other memory chip or cartridge, and networked versions of the same. The one or more processors can include any combination of any number of a central processing unit (CPU), a graphics processing unit (GPU), a neural processing unit (NPU), a microcontroller unit (MCU), an optical processor, a programmable logic controller, a microcontroller, a microprocessor, a digital signal processor, an intellectual property (IP) core, a Programmable Logic Array (PLA), a Programmable Array Logic (PAL), a Generic Array Logic (GAL), a Complex Programmable Logic Device (CPLD), a Field-Programmable Gate Array (FPGA), a System On Chip (SoC), an Application-Specific Integrated Circuit (ASIC), or the like. In some embodiments, the one or more processors can also be a set of processors grouped as a single logical component.

In some embodiments, while the examples in the present disclosure are described herein with reference to retinal imaging, the various operational modes, such as the sparse mode, are adaptable to various hyperspectral imaging applications.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive.

It is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A hyperspectral imaging system comprising:
an illumination unit comprising:
a plurality of LEDs configured to emit light comprising at least one spectral band; and
a first optical assembly configured to receive the light from the plurality of LEDs and output a light beam comprising the at least one spectral band to illuminate a target region of an imaging subject;
an imaging unit comprising:
a second optical assembly configured to collect light reflected from the target region and direct the collected light to an image sensor; and
an image sensor configured to capture an image of the target region;
a control unit in communication with the illumination unit and the imaging unit, the control unit comprising at least one processor and a non-transitory storage medium storing instructions that, when executed by the at least one processor, cause the system to perform operations comprising:
activating at least one LED of the plurality of LEDs based on an activation pattern, wherein the activation pattern comprises a time sequence of selections of one or more LEDs; and
synchronizing the image capture of the target region and the illumination of the target region by coordinating exposure timing of the image sensor and the activation of the at least one LED.

2. The system of claim 1, wherein the activation pattern comprises a random or a pseudo-random selection of one or more LEDs from the plurality of LEDs.

3. The system of claim 2, wherein the random or pseudo random selection is based on a sparse sampling of a preselected spectral range.

4. The system of claim 1, wherein the first optical assembly comprises a spherical enclosure configured to collect the light emitted from the at least one LED, the spherical enclosure comprising:
an inner reflective surface; and
an output aperture configured to output the collected light.

5. The system of claim 1, wherein when two or more LEDs are activated at the same time, the spectral bands of the activated LEDs do not overlap.

6. The system of claim 1, wherein the operations further comprising reconstructing a hyperspectral dataset of the target region over a preselected spectral range, the hyperspectral dataset comprising a two-dimensional image for each wavelength or spectral band of the preselected spectral range.

7. The system of claim 1, wherein the target region comprises a portion of the retina.

8. The system of claim 7, wherein the operations further comprise:
obtaining a hyperspectral profile of the portion of the retina over the preselected spectral range; and
detecting a presence or absence of at least one spectral signature in the hyperspectral profile indicative of the presence or absence of a biomarker in the retina.

9. The system of claim 8, wherein the hyperspectral profile includes a reflectance spectral profile of the portion of the retina over the preselected spectral range.

10. The system of claim 9, wherein the operations further comprise:
measuring an output power or intensity of the at least one LED activated based on the activation pattern; and
determining the reflectance spectral profile of the portion of the retina over the preselected spectral range based on the measured output power or intensity.

11. A computer-implemented method for hyperspectral imaging, the method comprising:
activating at least one LED of a plurality of LEDs based on an activation pattern, wherein the plurality of LEDs are configured to emit light comprising at least one spectral band, and the activation pattern comprises a time sequence of selections of one or more LEDs;
capturing, by an image sensor, one or more images of a target region of an imaging subject under the illumination of the activated at least one LED; and
synchronizing the image capture of the target region and the illumination of the target region by coordinating exposure timing of the image sensor and the activation of the at least one LED.

12. The computer-implemented method of claim 11, wherein the activation pattern comprises a random or a pseudo-random selection of one or more LEDs from the plurality of LEDs.

13. The computer-implemented method of claim 12, wherein the random or pseudo random selection is based on a sparse sampling of a preselected spectral range.

14. The computer-implemented method of claim 11, further comprising collecting light emitted from the at least one LED using a spherical enclosure, the spherical enclosure comprising:
an inner reflective surface; and
an output aperture configured to output the collected light.

15. The computer-implemented method of claim 11, wherein when two or more LEDs are activated at the same time, the spectral bands of the activated LEDs do not overlap.

16. The computer-implemented method of claim 11, further comprising reconstructing a hyperspectral dataset of the target region over a preselected spectral range, the hyperspectral dataset comprising a two-dimensional image for each wavelength or spectral band of the preselected spectral range.

17. The computer-implemented method of claim 11, wherein the target region comprises a portion of the retina.

18. The computer-implemented method of claim 17, further comprising:
obtaining a hyperspectral profile of the portion of the retina over the preselected spectral range; and
detecting a presence or absence of at least one spectral signature in the hyperspectral profile indicative of the presence or absence of a biomarker in the retina.

19. The computer-implemented method of claim 18, wherein the hyperspectral profile includes a reflectance spectral profile of the portion of the retina over the preselected spectral range.

20. The computer-implemented method of claim 19, further comprising:
measuring an output power or intensity of the at least one LED activated based on the activation pattern; and
determining the reflectance spectral profile of the portion of the retina over the preselected spectral range based on the measured output power or intensity.
